(19) United States
(12) Patent Application Publication
(10) Pub. No.: US 2023/0121319 A1
(43) Pub. Date: Apr. 20, 2023

(54) Integrated Bedside Echocardiogram Monitor

(71) Applicant: Tzvi Neuman, Flushing, NY (US)

(72) Inventor: Tzvi Neuman, Flushing, NY (US)

(21) Appl. No.: 17/502,682

(22) Filed: Oct. 15, 2021

(51) Int. Cl. A61B 8/04; A61B 8/14; A61B 8/08; A61B 8/00; B06B 1/06

(52) U.S. Cl. CPC A61B 8/04; A61B 8/14; A61B 8/0883; A61B 8/4488; A61B 8/4477; A61B 8/463; B06B 1/0607

(57) ABSTRACT

A system and method are provided for transthoracic echocardiogram imaging using ultrasound transducer arrays. The arrays are distinctly grouped, focused, positioned and directed within a housing comprising a solid yet flexible pad suited for placement on a portion of a patient's body.

### TECHNICAL FIELD

The present application relates to ultrasound monitoring and devices for diagnosing and detecting certain cardiac conditions including shock.

### BACKGROUND

Shock is a dangerous medical condition that is marked by poor perfusion of the end-organ and can lead to organ failure or even death. There are several known causes and physiological and pathophysiological pathways to shock. It is important to identify the correct nature of the shock so as to apply the appropriate treatment including the proper medication. Failure to do so, or mis-diagnosing the cause or nature of the shock can lead to life-threatening complications or further deterioration of the condition of a patient or delay of recovery. The clinical staff treating a shock patient should quickly determine whether the shock is due to heart failure, heart obstruction, loss of blood or dilated vasculature, for example.

Existing modalities for evaluating shock include central venous pressure, pulmonary artery catheter, mixed venous sampling and lactic acid level tests. Other modalities may employ monitoring of systemic vascular resistance, cardiac output monitoring and monitoring the change in pulse blood pressure waveforms. These parameters are often difficult to monitor with accuracy and are not generally consistent across applications or real-world clinical settings.

One generally accepted method for treating shock patients is to flood the patient's system with fluid and observe the patient's response. However, some patients experience fluid overload as a result that requires diuresis, which is a short-term solution, but it can be detrimental to the patient's cardiac fitness in the long term. Therefore, in acute care settings that are properly staffed and equipped, it is sometimes preferable to actually observe (image) the patient's heart using echocardiography.

Transthoracic echocardiogram (TTE) is a medical modality for examination of cardiac conditions and generating image data of the heart using ultrasound energy. The user or clinician can observe the structure and function of the heart and related anatomy using TTE in a non-invasive manner (without cutting open or injuring the patient's body to make such observations). TTE has become a common tool for use in intensive care units (ICU) and clinical settings, especially to assess the condition of patients who have suffered shock or are going into shock.

TTE allows the clinical staff to quickly assess the patient's cardiac function and structure, bedside, in real time. Unfortunately, TTE requires highly trained staff and expert placement of the special cardiac ultrasound probe on the patient's thorax at specific locations, and further acquiring several images to make an appropriate clinical determination. If properly installed, operated and read, the results of TTE can allow necessary application of fluids, diuretics or vasoactive agents such as chronotropic, inotropic medications to treat a patient in shock. TTE is also used by emergency room (ER) staff, critical care medical staff, surgeons and other first responders such as battlefield medical staff, if suitably equipped and trained.

Known limitations of TTE include the availability of proper equipment, and the specialized training and knowledge required to conduct and process the TTE. Specifically, as mentioned above, the correct application of TTE requires specialized knowledge of the placement of the cardiac ultrasound probe on the patient's body. This knowledge is not always available in all settings, bedside, especially in emergency or field conditions or when intensive care units are operating at capacity. For example, some environments lack the necessary staff to interpret the readings of a TTE, at all hours, even if a TTE is available and properly applied to a patient. Other challenges include that existing TTE probes are bulky piezo electric crystal transducers (PZT) and difficult to properly apply to patients.

A problem therefore exists in the availability and proper administration and reading of a TTE in traditional settings using traditional TTE equipment.

### SUMMARY

One or more embodiments are directed to a transthoracic echocardiogram monitor comprising a processor-controlled ultrasound imaging unit having an outer solid yet flexible pad housing a plurality of geometrically curved ultrasound transducer arrays; each of said transducer arrays facing along a respective orientation and having a respective geometric focal distance, each of said transducer arrays further comprising a plurality of respective transducer elements configured and arranged to emit and to receive ultrasound energy at a determined center frequency of each respective transducer array, and wherein a focus of each respective transducer array is at said respective geometric focal distance of the respective array and along a respective orientation for said respective transducer array; wherein a first one of said transducer arrays is configured and arranged to have a first focal distance and a first orientation to scan a PLA image zone, and a second one of said transducer arrays is configured and arranged to have a second focal distance and a second orientation to scan a PSA image zone, wherein said first and second transducer arrays are disposed one above the other within a PLA/PSA position in said pad; and wherein a third one of said transducer arrays is configured and arranged to have a third focal distance and a third orientation to scan an A4C image zone; and wherein a fourth one of said transducer arrays is configured and arranged to have a fourth focal distance and a third orientation to scan a SC image zone.

Some embodiments are directed to a method for diagnosing shock, comprising disposing an ultrasonic imaging unit comprising a solid yet flexible pad containing a plurality of ultrasonic transducer arrays proximal to a region of interest; delivering, from each of said transducer arrays a respective set of focused ultrasound energy waves at a respective orientation and depth; wherein delivering said respective set of focused ultrasound energy waves from the respective transducer arrays comprises delivering a first set of focused ultrasound imaging waves to a PLA image zone within said region of interest, and a second set of focused ultrasound imaging waves to a PSA image zone within said region of interest, wherein said first and second transducer arrays are delivered from respective PLA and PSA arrays that are disposed one above the other within said pad; and further comprises delivering a third set of focused ultrasound energy waves to an A4C image zone within said region of interest; and further comprises delivering a fourth set of focused ultrasound energy waves one to a SC image zone within said region of interest.

### BRIEF DESCRIPTION OF THE DRAWINGS

Fora fuller understanding of the nature and advantages of the present invention, reference is made to the following detailed description of preferred embodiments and in connection with the accompanying drawings, in which:

FIG. 1 illustrates an exemplary block-level arrangement of a system according one or more embodiments;

FIG. 2 illustrates some functional blocks of an ultrasound imaging unit according to one or more embodiments;

FIG. 3 illustrates an exemplary arrangement and placement of ultrasound transducers of the ultrasound imaging unit;

FIG. 4 illustrates a cross section of an exemplary ultrasonic imaging apparatus; and

FIG. 5 illustrates exemplary placement of the ultrasound imaging unit.

### DETAILED DESCRIPTION

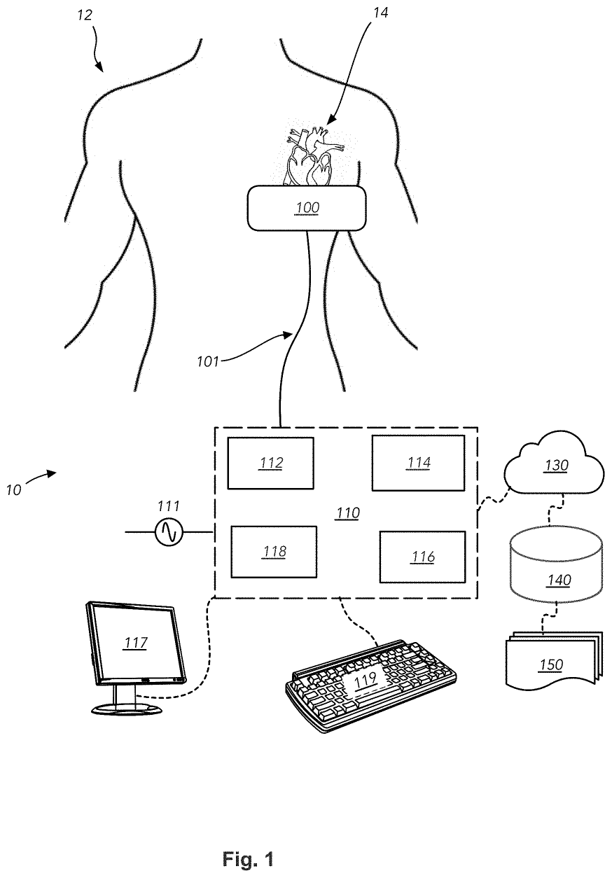

FIG. 1 illustrates an exemplary block-level arrangement of a system 10 according to the present invention that may be used on a patient 12 who has gone into shock, and more specifically to achieve transthoracic echocardiography of the heart 14 of the patient.

The system 10 includes an ultrasound imaging transducer unit 100 to be discussed in more detail below, a computing unit 110 comprising one or more processing circuits such as but not limited to a central processing unit (CPU) 112, waveform monitoring circuit 114, user and/or communication interface 116, digital storage or memory unit 118, display 117, keyboard 119, power supply 111 and other components as would be appreciated by those skilled in the art. However, the design and configuration, as well as the programmed data or machine-readable instructions of this and other preferred embodiments is unique and new and offers the features and limitations given herein with respect to this invention. The result is that the present processor-driven system 10 and novel ultrasound unit 100 render previously-unknown capabilities to simplify and make echocardiograms of patients in shock possible from the bedside, and further enable the use of this technology in situations where highly trained personnel may not be available to install, place and read conventional TTE. Various particular novel aspects of the design and operation of system 10 are addressed and illustrated throughout this disclosure.

In an aspect, the system 10 is equipped with a user interface or input/output (IO) interface and attachments which in one example include a keyboard 119 operable and coupled to processing unit 110 so as to allow entry of information and instructions that cause the system 10 to switch between desired views and image output modes displayed on display unit 117.

The display unit 117 may be any local or remote display device such as a computer monitor that can be specially provided for this purpose, or which can be part of an existing bedside or remote system that the current system and method may be switched to display.

In addition, the user interface can be configured to adjust and control the selected parameters such as depth (measured from the base of the probe to the distal or posterior aspect of the patient's heart), transducer gain, image zoom magnification, cursor position, image freeze and unfreeze (time snap shot), virtual caliper used to measure distances, selection of ultrasound Doppler and color image modes, control of selected electro cardiogram (EKG) waveform, and image save functions. In this way, operators or medical staff can configure and manipulate system 10 and computing processor unit 110 as well as ultrasound imaging unit 100.

The system 10 further includes or can be coupled to a communication interface as understood by those skilled in the art, which may exchange digital data, instructions and other control signals over a communication network 130 such as the Internet and/or wide area or local area networks. In an optional aspect, a database 140 coupled to system 10 or another server can be used to store patient record data and files 150. The components described may be configured in a number of ways as suits a given implementation without departing from the scope of the present invention. For example, some components may be divided into sub-units each having a distinct function or may be combined into one unit or circuit such as an integrated circuit. The interconnections between one or more components of system 10 can be achieved as best suits the given implementation. For example, the ultrasound imaging unit 100 may be coupled to the processing unit 110 with a hard-wired connection 101 having appropriate terminations. Other connections and data couplings may be achieved with wired, wireless or other means.

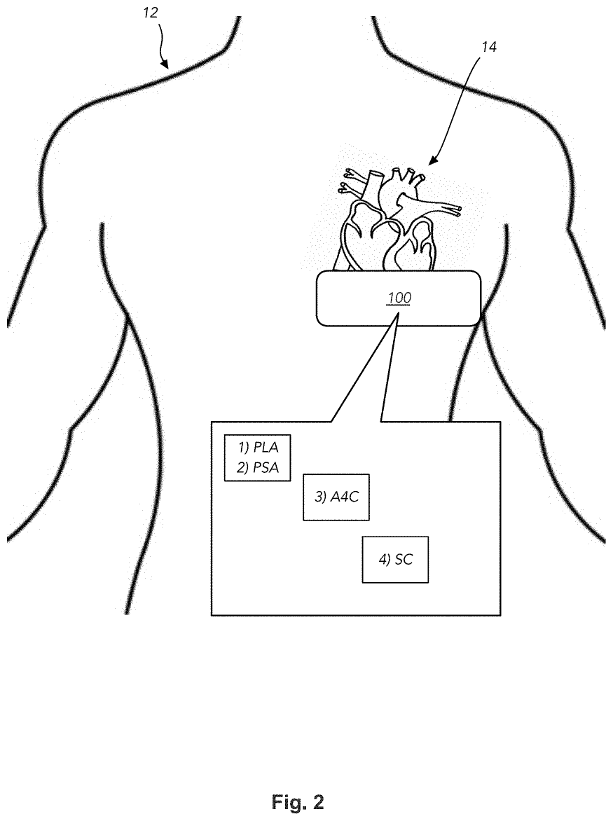

FIG. 2 illustrates some functional blocks of the ultrasound imaging unit 100 according to one or more embodiments. The unit may be configured and arranged to transmit and/or receive ultrasound energy pulses, waveforms and signals so as to generate an output signal representing one or more of: a cardiac parasternal short axis (PSA), parasternal long axis (PLA), subcostal (SC), and apical four-chamber view (A4C). In some cases, the ultrasound imaging unit 100 comprises hardware, transducer elements, arrays and signal carrying lines to perform focused cardiac ultrasound functions and deliver output signals representing the above images to the processing unit 110. Processing of said signals may be achieved within the main circuitry of the processing unit 110, and/or may be processed or pre-processed within the ultrasound imaging unit 100. The processing unit 110, along with the user interface components and instructions described herein permit the operator to control the imaging modality of the system 10 and switch between the above-mentioned representations as needed to achieve the required diagnostic goals.

More specifically, and without limitation but by way of illustration of preferred embodiments and modes, the system 10 in the foregoing imaging modes can provide and operate based on anatomical landmarks for TTE in the present context and in real-time on a patient from the bedside of the patient. For example (1) in the PLA mode the system manually and/or automatically selects the anatomical landmarks. The landmarks can be identified and marked or stored or referenced manually (by the operator using a landmark input marking device such as a cross hair or cursor) or achieved using an automated image processing or artificial intelligence (AI) means that recognizes such anatomical landmarks comprising the patient's left sternal border, in the left 3^{rd} to 4^{th} intercostal space, with the imaging transducer 100 oriented to the patient's right shoulder. An exemplary optimal depth setting is 12 to 24 cm for most cases; (2) in the PSA mode, the system selects the landmarks comprising the left sternal border, in the left 3^{rd} to 4^{th} intercostal space, with the imaging transducer 100 oriented to the patient's left shoulder. An exemplary optimal depth setting is 12 to 16 cm; (3) in the A4C mode, the imaging transducer 100 is placed on apical impulse with an orientation towards the 3 o'clock position. An exemplary optimal depth setting is 14 to 18 cm; and (4) in the SC mode, the ultrasound imaging probe 100 index is placed 2 to 3 cm below the xyphoid process to obtain a four chamber view with the probe direction oriented towards the patient's left shoulder, followed by a counterclockwise rotation to about the 12 o'clock position to obtain an IVC image and to determine respiratory variations of the IVC if needed. An exemplary optimal depth setting is 16 to 24 cm. The foregoing figures, sequence of rotation, dimensions and details are exemplary and those skilled in the art will understand when and how to extend them to other values. These examples are also approximate where quantified, such that the term “approximate” in herein allows for a 25% variation in either direction from a stated exemplary quantity or value.

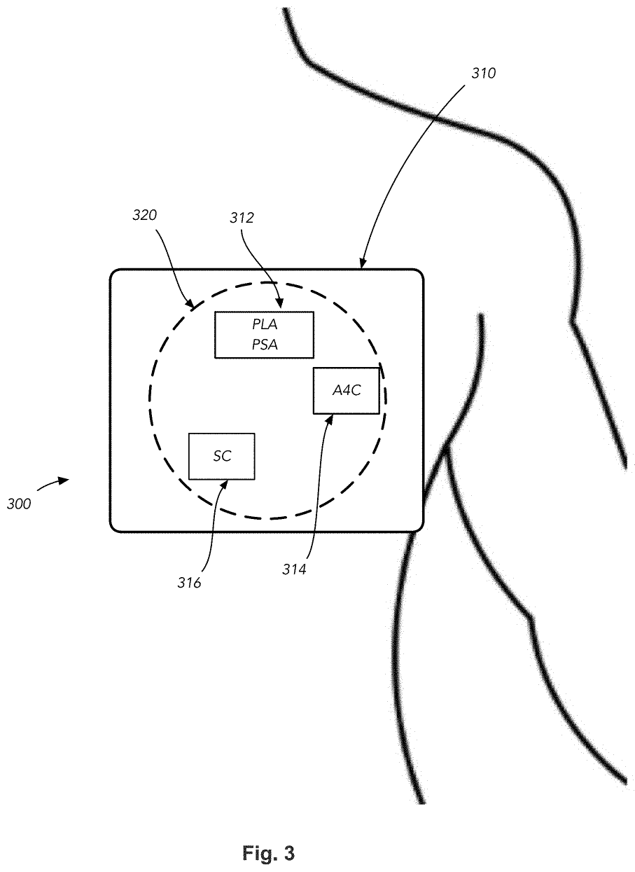

FIG. 3 illustrates an exemplary arrangement and placement of ultrasound transducers of the ultrasound imaging unit 300, having a pad 310 that comprises a solid yet flexible housing which can be positioned and formed on and around the region of interest 320, each imaging mode (PLA/PSA, A4C, SC) placed in a respective sub-zone or location. In an example, the PLA and PSA zones are located in a first location 312 of the region of interest 320; the A4C zone is located in a second location 314; and the SC zone 316 is located in a third location of the region of interest 320. The placement of the zones is preferably chosen to permit the sweeping of the image across the corresponding sub-zone using the landmarks described earlier as guidelines.

In an embodiment, the various zones are imaged using separate ultrasound imaging unit each having its own crystals. In another embodiment, the various zones are imaged using a same ultrasound imaging unit with multiple separate crystals each designed to be used with one of the imaging zones The crystals used for said imaging are preferably oriented along distinct respective orientations (e.g., directions in a plane substantially parallel to an imaged body, skin surface or anatomy of interest).

In an aspect, the respective imaging modes are easily set up by the operator in the present invention, as they are pre-positioned within the pad housing 310 of the imaging unit 300. This way, the operator can just place the pad 310 over the thoracic cavity of the patient, e.g., proximal to the heart, and the sub-zones are located within the region of interest 320 to automatically capture the respective image modes as selected.

In another aspect, the ultrasound unit's housing pad 310 is solid to secure and maintain the position of the internal components, yet pliable or flexible enough to allow wrapping or contouring over the patient's body and better acoustic transmission between the transducer and the patient's body without unnecessary air gaps. An ultrasonic coupling gel, liquid or pad layer can be further introduced between the ultrasound unit 300 and the patient's body to improve acoustic transmission of the ultrasonic imaging waves into and out of the region of interest 320. A power and signal transmission or communications interface and cable is used to connect the ultrasound imaging unit 300 to a processing and image display circuit and apparatus as described above. However, it is also possible to couple the ultrasound imaging unit 300 to said components using wireless communication links or networks such as Bluetooth, Wi-Fi, cellular communication, infra-red transmission, and so on.

In still another aspect, the ultrasonic transducer elements of the imaging unit preferably comprise one or more solid state piezoelectric micromachined ultrasound transducers that are compact and efficient. In an embodiment, the transducer elements are disposed in a curved phased array, or groups of curved phased arrays that are time-sequenced so as to provide pulse-echo image data corresponding to the structure of the patient's anatomy (e.g., heart and surrounding region of interest 320). These can be manually manipulated to sweep through the region of interest, for example between the landmarks as described above, e.g., using a cursor, joystick, computer mouse or trackball control device. The operator can thus obtain an image view of each zone exclusively.

In an embodiment, the PLA/PSA zone and the SC zone will require two sheets or layers of transducers, one on top of the other, with each sheet of transducer arrays aligned in a determined orientation in order to obtain the respective rotated views.

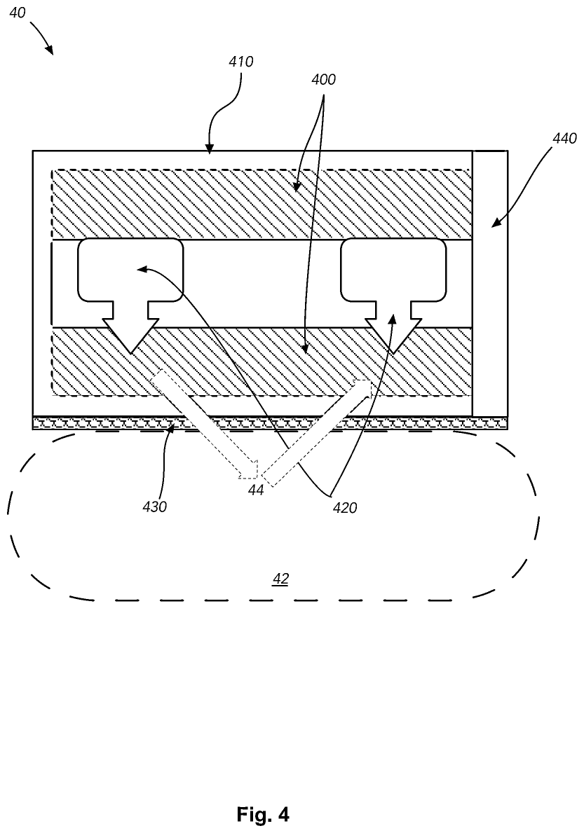

FIG. 4 illustrates a cross section of an exemplary ultrasonic imaging apparatus 40 for use in the present invention. The apparatus includes an outer hypoallergenic flexible shell or pad 410. A pair of ultrasonic transducers 400 are disposed within the outer pad 410. The transducers 400 may be in the form of planar slabs or sheets (i.e., having a thickness substantially thinner than their lateral extent, e.g., their thickness being one tenth to one fifth of their lateral dimension). The present drawings may not be to scale but are rather for the purpose of explanation. The pair of transducers 400 are disposed one above the other as shown. The PLA/PSA zone employs a pair (two) substantially parallel transducer plates or sheets as described, one above the other, each plate or sheet having a different alignment in order to acquire two respectively different images in the same region of interest or sub-zone, rotated by an angular difference between the two.

A pair of EKG leads 420 built into pad 410 are disposed between the transducers 400. The EKG leads may or may not contact the skin of a patient or the anatomy of interest and are represented in the figures for the sake of explanation. For example, the EKG leads may be embedded in or between imaging crystals, or in some aspects may be disposed within an electrically conductive pad that houses the transducers. An electrical and/or electronic signal and power and data interface 440 couples the unit 40 to the other components of the present system for delivery of power supply voltage to the transducers, and for delivery of ultrasound image signals and other data to a processing unit as mentioned earlier. The interface 440 may be in the form of a multi-pin connector. In addition, the pad 410 may include, or an additional layer of acoustically transmissive material 430 may be disposed beneath the apparatus to provide improved ultrasound transmission through coupling layer 430. Preferably, coupling layer 430 comprises a semi-solid gel, a liquid or another material having appropriate acoustic impedance characteristics to minimize ultrasound wave 44 absorption, loss or reflection at the interface between the imaging unit and the region of interest 42 of the patient's body.

The transducers and/or array elements may be arranged in layers disposed one above the other with respect to the anatomy of interest or patient's body, e.g., in planes parallel to the patient's body surface. The transducers and/or array elements may alternatively be in the same plane as one another but tiled so that they each occupy a respective separate area within said same plane.

In a non-limiting aspect, the transducer elements have a center frequency (are most efficiently configured to send and receive) between 2.5 MHz and 3.5 MHz, by design, so that the quality and penetration depth and power of the unit is optimized for imaging the region of interest and cardiac and thoracic volumes of a typical patient.

In another non-limiting aspect, the transducers are arranged into arrays (multiple individually powered transducer elements) that are geometrically disposed along a curved profile (having a design radius of curvature) so as to provide a geometric focus at a depth (distance from the transducer face) of about 28 cm. Again, the examples provided herein are non-limiting preferred embodiments, and the scope of the invention is not limited by such examples. This arrangement can provide two-dimensional (2D) echo images of the region of interest including for example a patient's thoracic or cardiac volume and anatomy.

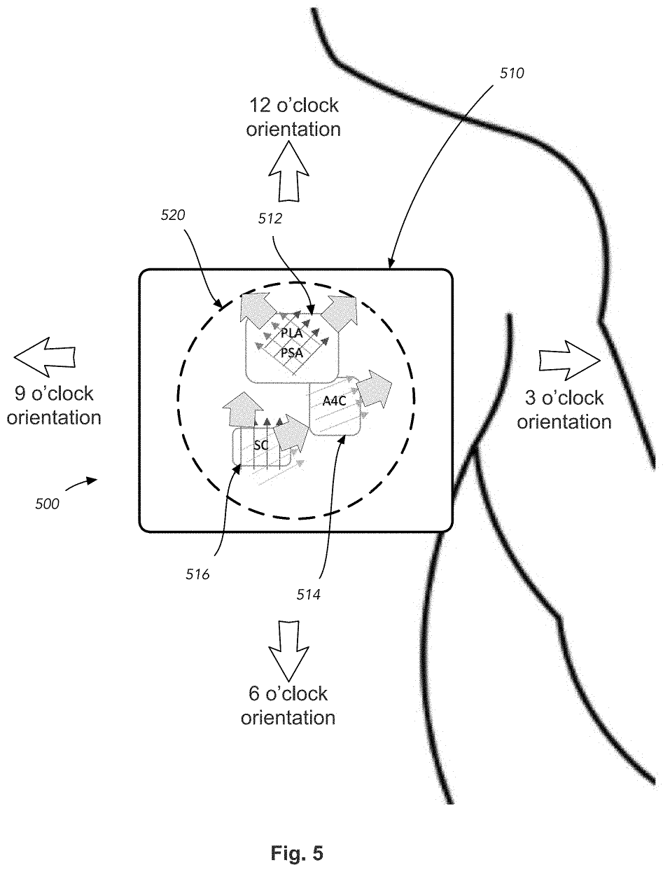

FIG. 5 illustrates exemplary placement of the ultrasound imaging unit 500 with respect to an anterior view of a patient. The figure illustrates the alignment of transducers within the ultrasound imaging unit 500 and pad 510 described earlier. We see that the PLA/PSA zones 512 are equipped with two different transducer orientations (depicted by arrow directions); the A4C transducer has a given orientation; and the SC zone transducer has another given orientation as described before in respective zones 514 and 516. The optimal placement of the ultrasound pad 510 with respect to the patient best employs the curved phased arrays and orients the transducers according to the imaging modes needed. An “index point” or reference position may be defined that describes the orientation of the transducer arrays and PZT crystal structure to generate a corresponding 2D image of the underlying anatomical structures. The ultrasound probe uses a clock face representation to target the TTE device and obtain the desired image within the volume, region of interest 520. As mentioned elsewhere, the PLA transducer with the probe indicator directed towards the patient's right shoulder as shown may be set for a depth of approximately 12 to 24 cm; the PSA (second layer) transducers is positioned in the same location as the PLA with the transducer oriented towards the patient's left shoulder and set for a depth of approximately 12 to 16 cm.

For best image acquisition, the operator may manually compress the pad and transducer assemblies against the patient's body (to obtain compressibility information about an anatomy), or may apply a force at an angle to the patient's body, e.g., angling with respect to the patient's body, to orient the image volume at a desired orientation, e.g., from apex to papillary muscles in the heart.

By proper angling of the transducers or probe in one or more degrees of freedom the system can obtain a more complete assessment of the heart organ at several anatomical levels: the aortic level, the mitral valve, the papillary muscles at mid-ventricle level and the apex. This angling can be accomplished at the bedside by the operator pressing down on the given position of the ultrasound pad 510.

The A4C transducer may be oriented towards the left side of the body at approximately 3 o'clock with a depth set for approximately 14 to 18 cm. In some instances, the angling of the transducer allows for a five-chamber view relating to the structure of the aortic valve or left ventricular outflow track, which may be achieved by the bedside operator manually pressing down at the appropriate location on the pad 510.

As mentioned before, the SC zone transducers are preferably placed 2 to 3 cm below the xyphoid process and directed towards the patient's left shoulder with an approximate depth set to 16 to 24 cm. In addition, subcostal IVC may be examined for respiratory variation, in which case the second layer of transducer plates or sheets is employed and oriented towards the 12 o'clock direction.

The orientation or directionality is illustrated in FIG. 5 by the respective gray arrows and can be defined against an anterior view of a patient's body where the patient's head is up (towards the 12 o'clock position) and the patient's left side is to the right in the diagrams (towards the 3 o'clock position) and the patient's feet are down (towards the 6 o'clock position) and the patient's right side is to the left in the diagrams (towards the 9 o'clock position). This directional orientation and positioning of a transducer array within the ultrasound imaging pad can be referred to as an index point for that array.

In one or more embodiments, a user interface includes a computer monitor or visual display unit 117 that can be controllably switched between display modes corresponding to the one or more imaging zones showing PLA, PSA, A4C and SC. In some embodiments the display can additionally provide a subcostal IVC view.

The user interface in some embodiments is adapted and configured to receive input signals (e.g., touch screen controls, manual button presses, drop down menus or similar interface elements) to permit one or more system parameter settings such as: operator control between multiple visual views of the imaging unit, depth control, gain, zoom magnification, cursor or arrow movement on screen, freeze and unfreeze of an image snapshot in real-time, virtual caliper to measure distances, color and Doppler mode, display of cardiac EKG waveform on screen, saving an image to a data storage unit, sending and saving data to an electronic medical record (EMR) database.

It is again noted that several examples and preferred embodiments are set forth above. These are not given by way of limitation, but instead to best illustrate the invention and disclosed systems and methods. Those skilled in the art will understand upon reviewing the present disclosure that some components or steps may be optional and omitted, others may be added, without loss of generality. Equivalent or substitute components or steps may also be applied, remaining within the scope of the invention captured by the disclosure.

What is claimed is:

1. A transthoracic echocardiogram monitor comprising:
a processor-controlled ultrasound imaging unit having an outer solid yet flexible pad housing a plurality of geometrically curved ultrasound transducer arrays;
each of said transducer arrays facing along a respective orientation and having a respective geometric focal distance, each of said transducer arrays further comprising a plurality of respective transducer elements configured and arranged to emit and to receive ultrasound energy at a determined center frequency of each respective transducer array, and wherein a focus of each respective transducer array is at said respective geometric focal distance of the respective array and along a respective orientation for said respective transducer array;
wherein a first one of said transducer arrays is configured and arranged to have a first focal distance and a first orientation to scan a PLA image zone, and a second one of said transducer arrays is configured and arranged to have a second focal distance and a second orientation to scan a PSA image zone, wherein said first and second transducer arrays are disposed one above the other within a PLA/PSA position in said pad;
and wherein a third one of said transducer arrays is configured and arranged to have a third focal distance and a third orientation to scan an A4C image zone; and
wherein a fourth one of said transducer arrays is configured and arranged to have a fourth focal distance and a third orientation to scan a SC image zone.

2. The monitor of claim 1, further comprising an electrical interface between said ultrasound imaging unit and a connected processor.

3. The monitor of claim 1, further comprising an acoustic interface layer with ultrasonic transmission and impedance properties matched to that of human tissue.

4. The monitor of claim 1, wherein the first (PLA) transducer array has a focal distance between 12 and 24 cm.

5. The monitor of claim 1, wherein the second (PSA) transducer array has a focal distance between 12 and 16 cm.

6. The monitor of claim 1, wherein the third (A4C) transducer array has a focal distance between 14 and 18 cm.

7. The monitor of claim 1, wherein the fourth (SC) transducer array has a focal distance between 16 and 24 cm.

8. The monitor of claim 1, wherein said center frequency of said transducer elements is between 2.5 MHz and 3.5 MHz.

9. The monitor of claim 1, further comprising a display screen configured and arranged to receive and to display a plurality of image views corresponding to respective one or more image zones of said monitor.

10. The monitor of claim 1, wherein said transducer elements comprise solid state piezoelectric micromachined ultrasound transducer elements.

11. A method for diagnosing shock, comprising:
disposing an ultrasonic imaging unit comprising a solid yet flexible pad containing a plurality of ultrasonic transducer arrays proximal to a region of interest;
delivering, from each of said transducer arrays a respective set of focused ultrasound energy waves at a respective orientation and depth;
wherein delivering said respective set of focused ultrasound energy waves from the respective transducer arrays comprises delivering a first set of focused ultrasound imaging waves to a PLA image zone within said region of interest, and a second set of focused ultrasound imaging waves to a PSA image zone within said region of interest, wherein said first and second transducer arrays are delivered from respective PLA and PSA arrays that are disposed one above the other within said pad; and further comprises delivering a third set of focused ultrasound energy waves to an A4C image zone within said region of interest; and further comprises delivering a fourth set of focused ultrasound energy waves one to a SC image zone within said region of interest.

12. The method of claim 11, further comprising applying hemodynamic monitoring to generate a decision based on said hemodynamic monitoring and said delivery of the focused ultrasound imaging waves.
